(19) United States
(12) Patent Application Publication
(10) Pub. No.: US 2023/0355501 A1
(43) Pub. Date: Nov. 9, 2023

(54) COSMETIC COMPOSITION

(71) Applicant: AJINOMOTO CO., INC., Tokyo (JP)

(72) Inventors: Fumie Ookura, Kanagawa (JP); Yoshinobu Takino, Kanagawa (JP); Kenichi Mori, Kanagawa (JP); Shingo Nakanuma, Kanagawa (JP); Eriko Iwasaki, Kanagawa (JP); Masashi Suzuki, Kanagawa (JP); Daishi Sakaguchi, Kanagawa (JP)

(73) Assignee: AJINOMOTO CO., INC., Tokyo (JP)

(21) Appl. No.: 18/354,129

(22) Filed: Jul. 18, 2023

Related U.S. Application Data
(63) Continuation of application No. PCT/JP2022/002032, filed on Jan. 20, 2022.

(30) Foreign Application Priority Data
Jan. 20, 2021 (JP) 2021-006808

(51) Int. Cl. A61K 8/68; A61Q 19/00

(52) U.S. Cl. CPC A61K 8/68; A61Q 19/00

(57) ABSTRACT

A composition, such as a cosmetic composition, is provided. The composition, such as a cosmetic composition, contains two or more kinds of phytoceramides (PHCs) having different lengths of alkyl chains, for example, in specific amounts.

This application is a Continuation of, and claims priority under 35 U.S.C. § 120 to, International Application No. PCT/JP2022/002032, filed Jan. 20, 2022, and claims priority therethrough under 35 U.S.C. § 119 to Japanese Patent Application No. 2021-006808, filed Jan. 20, 2021, the entireties of which, as well as all citations cited herein, are incorporated by reference herein.

### BACKGROUND

### Technical Field

The present invention relates to a composition, such as a cosmetic composition.

### Background Art

A ceramide is a compound having a structure in which a sphingoid base and a fatty acid are covalently linked to each other via an amide bond. That is, a ceramide includes a sphingoid base moiety (i.e. an alkyl chain) and a fatty acid moiety (i.e. an acyl chain), which moieties are covalently linked to each other via an amide bond. Various groups of ceramides have been known depending on the types of the sphingoid base and fatty acid (see Masukawa Y. et al., J Lipid Res. 2008 July; 49(7):1466-76). Some ceramides have been used as ingredients for pharmaceuticals, cosmetics, and so forth. Phytoceramide (PHC) is a ceramide of phytosphingosine (PHS). That is, PHC is a ceramide in which the sphingoid base moiety is PHS. That is, PHC has a PHS moiety (i.e. an alkyl chain) and a fatty acid moiety (i.e. an acyl chain), which moieties are covalently linked to each other via an amide bond. As PHC, variant species having various different lengths of alkyl chains and various different lengths of acyl chains have been known (Masukawa Y. et al., J Lipid Res. 2008 July; 49(7):1466-76). There has been reported a composition containing two or more kinds of PHC species having different lengths of acyl chains (see WO2008/043386 and Oh M J. et al., Clin Cosmet Investig Dermatol. 2017 Sep. 13; 10:363-371). However, there has been no reports of any composition containing two or more kinds of PHC species having different lengths of alkyl chains.

### SUMMARY

An aspect of the present invention is to provide a composition, such as cosmetic composition.

Provided herein is the finding that the combined use of two or more kinds of PHC species having different lengths of alkyl chains improves a property of a composition containing them.

It is an aspect of the present invention to provide a composition, comprising two or more phytoceramides each having different lengths of alkyl chains.

It is a further aspect of the present invention to provide the composition as described above, wherein the two or more phytoceramides consist of:
(A) a phytoceramide (A) having a C16 phytosphingosine moiety in an amount of X % by weight to the total amount of the phytoceramides (A), (B), and (C);
(B) a phytoceramide (B) having a C18 phytosphingosine moiety in an amount of Y % by weight to the total amount of the phytoceramides (A), (B), and (C);
(C) a phytoceramide (C) having a C20 phytosphingosine moiety in an amount of Z % by weight to the total amount of the phytoceramides (A), (B), and (C);
wherein at least two of X, Y, and Z are more than 0, and the sum of X, Y, and Z are less than 100.

It is a further aspect of the present invention to provide the composition as described above, wherein the composition is a cosmetic or pharmaceutical composition.

It is a further aspect of the present invention to provide the composition as described above, wherein the composition further contains an ingredient typically used for formulating a cosmetic or pharmaceutical.

It is a further aspect of the present invention to provide the composition as described above, wherein the ingredient typically used for formulating a cosmetic or pharmaceutical is selected from the group consisting of an excipient, binder, disintegrant, lubricant, stabilizer, diluent, surfactant, pH adjuster, vitamin, mineral, perfume, pigment, preservative, terpenoid, lipid, fatty acid, alcohol, water, antioxidant, anti-inflammatory, moisturizing ingredient, anti-ageing ingredient, anti-cellulite ingredient, skin whitening ingredient, skin tanning ingredient, UV filter, and combinations thereof.

It is a further aspect of the present invention to provide the composition as described above, wherein the ingredient typically used for formulating a cosmetic or pharmaceutical is selected from the group consisting of a terpenoid, lipid, fatty acid, and combinations thereof.

It is a further aspect of the present invention to provide the composition as described above, wherein the terpenoid is selected from the group consisting of cholesterol, cholesterol sulfate, squalene, pristane, and combinations thereof.

It is a further aspect of the present invention to provide the composition as described above, wherein the lipid is selected from the group consisting of triolein, phosphatidylethanolamine, and combinations thereof.

It is a further aspect of the present invention to provide the composition as described above, wherein the fatty acid is selected from the group consisting of lauric acid, myristic acid, palmitic acid, stearic acid, arachidic acid, behenic acid, lignoceric acid, oleic acid, linolenic acid, and combinations thereof.

It is a further aspect of the present invention to provide the composition as described above, wherein X is 0-90, Y is 0-95, and Z is 0-50.

It is a further aspect of the present invention to provide the composition as described above, wherein X is 65-90, Y is 5-30, and Z is 1-20.

It is a further aspect of the present invention to provide the composition as described above,
wherein the phytoceramide (A) comprises a phytoceramide having a C16:0 phytosphingosine moiety,
wherein the phytoceramide (B) comprises a phytoceramide having a C18:0 phytosphingosine moiety, and
wherein the phytoceramide (C) comprises a phytoceramide having a C20:0 phytosphingosine moiety.

It is a further aspect of the present invention to provide the composition as described above,
wherein the phytoceramide (A) has an acyl chain having a length of C14 to C26,
wherein the phytoceramide (B) has an acyl chain having a length of C14 to C26, and
wherein the phytoceramide (C) has an acyl chain having a length of C14 to C26.

### BRIEF DESCRIPTION OF DRAWINGS

The FIGURE shows the results of the measurement of transepidermal water loss (TEWL).

### DETAILED DESCRIPTION OF THE EXEMPLARY EMBODIMENTS

<1> Composition

The composition as described herein is a composition containing two or more kinds of phytoceramides (PHCs) having different lengths of alkyl chains. These phytoceramides are also collectively referred to as “active ingredients”.

The composition may be, for example, a cosmetic composition or a pharmaceutical composition. The composition may particularly be a cosmetic composition. A cosmetic composition refers to a composition for cosmetic use, and may be synonymous with a cosmetic. A pharmaceutical composition refers to a composition for pharmaceutical use, and may be synonymous with a pharmaceutical. Both the terms “cosmetic composition” and “pharmaceutical composition” may include quasi drugs such as medicinal cosmetics, unless otherwise stated.

The composition can be used by, for example, applying the same to an organism. The phrase “application of the composition to an organism” may mean that the composition is brought into contact with a desired portion of an organism so that a desired effect is obtained. The composition can be applied to an organism in a manner described in the method as described below. The application target and application portion of the composition can be appropriately selected according to various conditions such as intended purpose of the composition. Examples of the organism, i.e. the application target of the composition, include mammals. Examples of the mammals include primates such as human, monkey, and chimpanzee; rodents such as mouse, rat, hamster, and guinea pig; and other various mammals such as rabbit, horse, cow, sheep, goat, pig, dog, and cat. Particular examples of mammals include human. Examples of the application portion of the composition include a body surface of an organism. Examples of the body surface include skin and body hair. Particular examples of the body surface include skin. The skin may be any part of skin. Examples of the skin include skin of face, head, and body. Examples of body include hands, arms, foots, legs, shoulders, breasts, waist, hip, back, and any other parts of body. The body hair may be any part of body hair. Examples of the body hair include head hair.

The organism may be a healthy individual or may be an unhealthy individual. The organism may or may not be, for example, an individual for which deterioration in condition of a body surface, such as skin and body hair, is observed. The organism may or may not be, specifically, for example, an individual having a symptom relating to deterioration in condition of a body surface, such as skin and body hair.

The application target of the composition may be in healthy state or may be in an unhealthy state. The application target of the composition may or may not be, for example, a body surface, such as skin and body hair, for which deterioration in condition is observed. The application target of the composition may or may not be, specifically, for example, a body surface, such as skin and body hair, having a symptom relating to deterioration in condition.

Examples of the “desired effect” when using the composition include an effect obtained from, e.g. application to an organism, of the active ingredients. That is, the effect obtained by use, e.g. application to an organism, of the active ingredients may be obtained by use, e.g. application to an organism, of the composition. In other words, the composition may have a function of providing the effect obtained by use, e.g. application to an organism, of the active ingredients. The aforementioned descriptions concerning use, e.g. application to an organism, of the composition can be applied similarly to use, e.g. application to an organism, of the active ingredients.

By using the active ingredients, specifically by applying the active ingredients to an organism, condition of the application target of the active ingredients may be improved, i.e. an effect of improving condition of the application portion of the active ingredients may be obtained. Hence, by using the composition, specifically by applying the composition to an organism, condition of the application target of the active ingredients, specifically, the application target of the composition, may be improved, i.e. an effect of improving condition of the application target of the active ingredients, specifically, the application target of the composition, may be obtained. In other words, the composition may have a function of improving condition of the application target of the active ingredients, specifically, the application target of the composition. Hence, the composition may be a composition for improving condition of the application target, such as a cosmetic or pharmaceutical composition for improving condition of the application target.

Examples of the improvement in condition include improvement in condition of a body surface, such as skin and body hair. Particular examples of the improvement in condition include improvement in skin condition. Hence, the composition may improve the condition of a body surface, such as skin and body hair, such as a cosmetic or pharmaceutical composition for improving condition of a body surface, such as skin and body hair. The composition may be, particularly, a composition for improving skin condition, such as a cosmetic or pharmaceutical composition for improving skin condition.

Examples of improvement in skin condition include improvement in skin lamellar structure (lamellar structure of skin), such as increase in orderliness of skin lamellar structure, improvement, such as increase, in skin barrier function (barrier function of skin), and improvement, such as increase, in skin moisturizing capacity (moisturizing capacity of skin). Hence, the composition may improve skin lamellar structure, skin barrier function, or skin moisturizing capacity, such as a cosmetic or pharmaceutical composition for improving skin lamellar structure, improving skin barrier function, or improving skin moisturizing capacity. Only one of these indices may be improved, or two or more of these indices may be improved. Improvement in a certain index may improve another index. For example, improvement in skin lamellar structure may improve a skin function such as skin barrier function and skin moisturizing capacity. In other words, a composition for improving skin barrier function or improving skin moisturizing capacity may be an embodiment of a composition for improving skin lamellar structure. Also, for example, improvement in skin barrier function may improve skin moisturizing capacity. In other words, a composition for improving skin moisturizing capacity may be an embodiment of a composition for improving skin barrier function. Specific examples of skin condition include condition of stratum corneum of skin. That is, improvement in skin lamellar structure may specifically be improvement in lamellar structure of stratum corneum of skin. Improvement in skin barrier function may specifically be improvement in barrier function of stratum corneum of skin. Improvement in skin moisturizing capacity may specifically be improvement in moisturizing capacity of stratum corneum of skin.

Methods for measuring improvement in condition of a body surface, such as skin and body hair, are not particularly limited. Improvement in condition of a body surface can be measured by using a parameter reflecting the condition of the body surface. For example, examples of parameters reflecting skin condition include transepidermal water loss (TEWL) and order parameter S. That is, improvement in skin condition can be measured as, for example, a decrease in TEWL. TEWL can be measured in a conventional manner. A decrease in TEWL may mean, specifically, for example, improvement in skin barrier function. A decrease in TEWL may also mean, specifically, for example, improvement in skin moisturizing capacity. Improvement in skin condition can also be measured as, for example, an increase in order parameter S. Order parameter can be measured in a conventional manner. Order parameter can be measured by, for example, the Electron Spin Resonance (ESR) method. Order parameter can be measured, specifically, for example, as described in the Examples section. Order parameter represents orderliness of array of fatty acid chains, such as orderliness of lamellar structure, and a higher value of order parameter S indicates that fatty acid chains are arrayed in a more orderly manner. An increase in order parameter S may mean, specifically, for example, improvement in skin lamellar structure.

It is sufficient that such indices as exemplified above each are improved when using the active ingredients, specifically, in case of using the composition as described herein, as compared with when not using the active ingredients, specifically, when not using the composition. The term “improvement” used for each of such indices as exemplified above shall include, when deterioration in this index occurs when not using the active ingredients, specifically, when not using the composition, that such deterioration is relieved when using the active ingredients, specifically, when using the composition.

By using the active ingredients, specifically by applying the active ingredients to an organism, an effect based on improvement in condition of the application target of the active ingredients may be obtained. It is expected that improvement in condition of the application target of the active ingredients enables preventing and/or treating a symptom relating to deterioration in condition of this target. That is, examples of the effect based on improvement in condition of the application target of the active ingredients include an effect of preventing and/or treating a symptom relating to deterioration in condition of this target. The symptom relating to deterioration in condition of the application target of the active ingredients may or may not be a result of disease. Examples of the symptom relating to deterioration in condition of the application target of the active ingredients include a symptom caused by deterioration in condition of this target and a symptom accompanied with deterioration in condition of this target. Hence, the composition may be used for preventing and/or treating a symptom relating to deterioration in condition of the application target thereof, such as a cosmetic or pharmaceutical composition for preventing and/or treating a symptom relating to deterioration in condition of the application target thereof.

Examples of the deterioration in condition include deterioration in condition of a body surface, such as skin and body hair. Particular examples of the deterioration in condition include deterioration in skin condition. Hence, the composition may be a composition for preventing and/or treating a symptom relating to deterioration in condition of a body surface, such as skin and body hair, such as a cosmetic or pharmaceutical composition for preventing and/or treating a symptom relating to deterioration in condition of a body surface, such as skin and body hair. The composition may be, particularly, a composition for preventing and/or treating a symptom relating to deterioration in skin condition, such as a cosmetic or pharmaceutical composition for preventing and/or treating a symptom relating to deterioration in skin condition.

Examples of deterioration in skin condition include deterioration in skin lamellar structure, such as decrease in orderliness of skin lamellar structure, deterioration, such as decrease, in skin barrier function, and deterioration, such as decrease, in skin moisturizing capacity. Examples of the symptom relating to deterioration in skin condition include rough skin, dry skin, squama (scale), desquamation, dermatitis, and psoriasis.

The active ingredients may be used for therapeutic purpose, or may be used for non-therapeutic purpose. That is, such effects as exemplified above each may be obtained for therapeutic purpose or non-therapeutic purpose, unless otherwise stated. In case of non-therapeutic purpose, the phrase “treatment of a symptom” may be interpreted as “improvement of a symptom”. The term “therapeutic purpose” may mean, for example, that an act on a human body for treatment is included, and particularly, may mean that an act is carried out as a medical act. The term “non-therapeutic purpose” may mean, for example, that an act on a human body for treatment is not included, and particularly, may mean that an act is carried out as a non-medical act. Examples of the non-therapeutic purpose include such purposes as those for health promotion and beauty.

By using the active ingredients in combination, a property of the composition can be improved as compared with when using either one of the active ingredients alone, that is, an effect of improving a property of the composition is obtained. This effect is also referred to as “effect of combined use of the active ingredients”. That is, for example, the effect of combined use of the active ingredients is obtained in the composition. Examples of the property include physical, chemical, and physiological properties. Specific examples of improvement in the property include a decrease in the melting point of the composition, and an increase in the lamellar structure-forming ability of the composition. Specific examples of improvement in the property also include an increase in the function of providing the effect obtained by use of the active ingredients. Examples of such a function include a function of improving condition of the application portion of the active ingredients, specifically, the application target of the composition, and a function of preventing and/or treating a symptom relating to deterioration in condition of the application target of the active ingredients, specifically, the application portion of the composition. An increase in the lamellar structure-forming ability of the composition can be measured as, for example, an increase in occurrence of the Maltese cross pattern. The Maltese cross pattern can be observed as described in the Examples section. A combined use of the active ingredients may provide only one of these effects, or may provide two or more of these effects. A certain effect may provide another effect. For example, an increase in the lamellar structure-forming ability may increase the function of providing the effect obtained by use of the active ingredients.

Phytoceramide (PHC) is a ceramide of phytosphingosine (PHS). PHC may also be referred to as, for example, “ceramide 3” or “ceramide NP”. Each variation of PHC is also referred to as “PHC species”. Each variation of PHS is also referred to as “PHS species”.

The term “phytosphingosine (PHS)” refers to a long-chain amino alcohol referred to as a sphingoid base, which has such a structure as described below. PHS includes an alkyl chain having an amino group at C2. That is, the carbon present at either one terminus of the alkyl chain and linked to the aminated carbon (position C2) is regarded as position C1 of the alkyl chain. The alkyl chain has two or more hydroxyl groups. The alkyl chain may have hydroxyl groups, for example, at C1, C3, and C4. The alkyl chain may or may not have additional hydroxyl group(s) other than the hydroxyl groups at C1, C3, and C4. The alkyl chain may typically have no additional hydroxyl group other than the hydroxyl groups at C1, C3, and C4. The length and the degree of unsaturation of the alkyl chain may vary. The alkyl chain may have a length of, for example, C14 to C26, such as C14, C16, C18, C20, C22, C24, and C26. The alkyl chain may have a length of, for example, particularly, C16, C18, or C20. The length of the alkyl chain may be interpreted as the carbon number, that is, the number of carbon atoms, of the alkyl chain. The alkyl chain may be saturated or unsaturated. The alkyl chain may have one or more unsaturated double bonds. That is, the term “alkyl chain” used for PHS and PHC is not limited to saturated chains, but may also include unsaturated chains, such as alkenyl and alkadienyl chains, unless otherwise stated. The alkyl chain may typically have no or only one unsaturated double bond. The alkyl chain may more typically have no unsaturated double bond. The alkyl chain may have, for example, a C8-trans double bond. The configurations of chiral centers may or may not be identical to those in the PHS moiety of a natural PHC. The position C2 may be, for example, 2S. The position C3 may be, for example, 3S. The position C4 may be, for example, 4R. The configurations of chiral centers may be, particularly, for example, 2S, 3S, and 4R. The number of carbons in the alkyl chain of PHS can be indicated as “n”. PHS having an alkyl chain of which the number of carbons is “n” is also referred to as “Cn PHS” or “Cn-alkyl PHS”. For example, the term “C18 PHS” collectively refers to PHS species having an alkyl chain having a length of C18, which may be saturated or unsaturated. The number of unsaturated double bonds in the alkyl chain of PHS can be indicated as “m”. PHS having an alkyl chain of which the number of carbons is “n” and the number of unsaturated double bonds is “m” is also referred to as “Cn:m PHS” or “Cn:m-alkyl PHS”. Examples of PHS can include such variant species of PHS, wherein the variant species have different lengths and/or different degrees of unsaturation. Specific examples of variant species of PHS include C16:0 PHS, which has a saturated C16 alkyl chain; C18:0 PHS, which has a saturated C18 alkyl chain; C20:0 PHS, which has a saturated C20 alkyl chain; C18:1 PHS, which has a C18 alkyl chain having one unsaturated double bond; and C20:1 PHS, which has a C20 alkyl chain having one unsaturated double bond. More specific examples of variant species of PHS include C16:0 PHS, C18:0 PHS, C20:0 PHS, C18:1 PHS, and C20:1 PHS, none of which have any additional hydroxyl group other than the hydroxyl groups at C1, C3, and C4. Examples of variant species of PHS may also include adducts of PHS, such as 4-(hydroxymethyl)-2-methyl-6-tetradecanyl-1,3-oxazinan-5-ol and 4-(hydroxymethyl)-2-methyl-6-hexadecenyl-1,3-oxazinan-5-ol, which may be generated via a reaction of either C18:0 PHS and C20:0 PHS with acetaldehyde, respectively. The term “phytosphingosine (PHS)” is not limited to C18:0 PHS, which is a typical species of PHS, but may collectively refer to variant species of PHS, such as C16:0 PHS, C18:0 PHS, C20:0 PHS, C18:1 PHS, and C20:1 PHS, or may collectively refer to such variant species of PHS and adducts thereof.

The term “phytoceramide (PHC)” refers to a compound including a structure of PHS covalently linked to a fatty acid via an amide bond. That is, PHC includes a PHS moiety (i.e. a moiety corresponding to PHS) and a fatty acid moiety (i.e. a moiety corresponding to a fatty acid), wherein the moieties are covalently linked to each other via an amide bond. The PHS moiety can also be referred to as an “alkyl chain”. The fatty acid moiety can also be referred to as an “acyl chain”. The amide bond may form between the amino group at C2 of PHS and a carboxyl group of the fatty acid. The aforementioned descriptions concerning PHS are similarly applicable to the PHS moiety of PHC. That is, for example, the length and the degree of unsaturation of the alkyl chain, that is, the PHS moiety, may vary as with those of PHS. That is, examples of PHC can include ceramides of the PHS species exemplified above. Specific examples of PHC include ceramides of C16 PHS, C18 PHS, and C20 PHS. More specific examples of PHC include ceramides of C16:0 PHS, C18:0 PHS, C20:0 PHS, C18:1 PHS, and C20:1 PHS. The length and the degree of unsaturation of the acyl chain, that is, the fatty acid moiety, of PHC may also vary. The acyl chain may have a length of, for example, C14 to C26, such as C14, C16, C18, C20, C22, C24, and C26. The acyl chain may have a length of, for example, particularly, C18. The length of the acyl chain may be interpreted as the carbon number, that is, the number of carbon atoms in the acyl chain. The acyl chain may be saturated, or may be unsaturated. The acyl chain may have one or more unsaturated double bonds. The acyl chain may or may not have a functional group (i.e. substituent group). The acyl chain may have one or more functional groups (substituent groups). Examples of the functional group (substituent group) include hydroxy group. The acyl chain may or may not have a hydroxy group, for example, at C2. The acyl chain may typically have no hydroxy group at C2. The carbon constituting the amide bond is regarded as position C1 of the acyl chain. The acyl chain may typically have no hydroxy group. The acyl chain may typically have no functional group (substituent group). PHC having an alkyl chain of which the number of carbons is “n”, that is, PHC with a Cn PHS moiety, can also be referred to as “Cn alkyl PHC”, “(phyto)ceramide of Cn PHS”, or “Cn PHS (phyto)ceramide”. The number of carbons in the acyl chain of PHC can be indicated as “x”. PHC having an acyl chain of which the number of carbons is “x”, that is, PHC with a Cx acyl moiety, can also be referred to as “Cx acyl PHC”. The number of unsaturated double bonds in the acyl chain of PHC can be indicated as “y”. PHC having an acyl chain of which the number of carbons is “x” and the number of unsaturated double bonds is “y”, that is, PHC with a Cx:y acyl moiety, can also be referred to as “Cx:y acyl PHC”. PHC having an alkyl chain of which the number of carbons is “n” and an acyl chain of which the number of carbons is “x”, that is, PHC with a Cn PHS moiety and a Cx acyl moiety, can also be referred to as “Cn alkyl/Cx acyl PHC”. For example, the term “C18 alkyl PHC”, “ceramide of C18 PHS”, or “C18 PHS ceramide” collectively refers to a PHC species having an alkyl chain with a length of C18 and having any acyl chain. For example, the term “C14 acyl PHC” collectively refers to a PHC species having an acyl chain with a length of C14 and having any alkyl chain. For example, the term “C18 alkyl/C14 acyl PHC” collectively refers to a PHC species having an alkyl chain with a length of C18 and an acyl chain with a length of C14. In case of PHC having an alkyl chain the number of carbons is “n” and the number of unsaturated double bonds is “m”, that is, PHC with a Cn:m PHS moiety, the term “Cn” used for the PHC name can be rewritten to “Cn:m”. The same shall apply to “Cx” of the acyl chain. For example, the term “C18:1 alkyl/C14:0 acyl PHC” collectively refers to a PHC species having an unsaturated alkyl chain with a length of C18 having one double bond and a saturated acyl chain with a length of C14. A PHC defined with the aforementioned name may be a single kind of PHC species, or may be a combination of two or more kinds of PHC species, unless otherwise stated. For example, a Cn PHS ceramide (Cn alkyl PHC) may be a single kind of Cn PHS ceramide, or may be a combination of two or more kinds of Cn PHS ceramides. Such a combination may be two or more kinds of Cn PHS ceramides having different alkyl chains and/or different acyl chains, such as alkyl chains having different degrees of unsaturation and/or acyl chains having different lengths and/or different unsaturation degrees. Also, for example, a Cn:m PHS ceramide may be a single kind of Cn:m PHS ceramide, or may be a combination of two or more kinds of Cn:m PHS ceramides. Such a combination may be two or more kinds of Cn:m PHS ceramides having different acyl chains, such as acyl chains having different lengths and/or different unsaturation degrees.

The combination of the active ingredients and the ratio of the active ingredients are not particularly limited, so long as an intended effect is obtained.

Examples of the combination of the active ingredients include a combination of two or all of C16 PHS, C18 PHS, and C20 PHS ceramides. That is, the active ingredients may be, for example, a combination of C16 PHS and C18 PHS ceramides, a combination of C16 PHS and C20 PHS ceramides, a combination of C18 PHS and C20 PHS ceramides, or a combination of C16 PHS, C18 PHS, and C20 PHS ceramides.

The amount of each active ingredient present in the composition, i.e. PHC having each length of the alkyl chain, to the total amount of the active ingredients, for example, may be 0.01% or more, 0.1% or more, 1% or more, 2% or more, 3% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, or 90% or more, by weight, may be less than 100, 99.99% or less, 99.9% or less, 99% or less, 97% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, or 10% or less, by weight, or may be within a range defined as a non-contradictory combination thereof. The amount of each active ingredient relative to the total amount of the active ingredients may specifically be, for example, 0.01-99.99%, 0.1-99.9%, 1-99%, 5-95%, 10-90%, 15-85%, 20-80%, 25-75%, 1-90%, 5-80%, 10-70%, 15-60%, 20-50%, 25-40%, 1-60%, 5-50%, 10-40%, 15-35%, or 20-30%, by weight. The amount of each active ingredient to the total amount of the active ingredients may also specifically be, for example, 0.01-10%, 0.1-10%, 1-10%, 10-20%, 20-30%, 30-40%, 40-50%, 50-60%, 60-70%, 70-80%, 80-90%, 90-99%, 90-99.9%, or 90-99.99%, by weight.

In other words, the composition may be, for example, a composition containing the PHCs (A), (B), and (C) shown below:
(A) PHC (A) having a C16 PHS moiety (i.e. C16 PHS ceramide) in an amount of X % by weight to the total amount of the PHCs (A), (B), and (C);
(B) PHC (B) having a C18 PHS moiety (i.e. C18 PHS ceramide) in an amount of Y % by weight to the total amount of the PHCs (A), (B), and (C);
(C) PHC (C) having a C20 PHS moiety (i.e. C20 PHS ceramide) in an amount of Z % by weight to the total amount of the PHCs (A), (B), and (C).

Two or all of X, Y, and Z are more than 0, and all of X, Y, and Z are less than 100. In other words, only one of X, Y, and Z may be 0. In this case, the PHCs (A), (B), and (C), specifically, the PHCs (A), (B), and (C) of which the amount is more than 0, are regarded as the active ingredients.

The amount of each of the PHCs (A), (B), and (C) in the composition, for example, may be 0% or more by weight, may be within the amount of each active ingredient exemplified above, or may be within a range defined as a non-contradictory combination thereof.

That is, each of X, Y, and Z, for example, may be 0 or more, 1 or more, 2 or more, 3 or more, 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, or 90 or more, may be less than 100, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, or 10 or less, or may be within a range defined as a non-contradictory combination thereof. X may specifically be, for example, 0-90, 1-85, 2-80, 65-90, 70-85, 5-20, or 1-15. Y may specifically be, for example, 0-95, 10-90, 15-85, 75-90, 50-65, 5-30, or 10-25. Z may specifically be, for example, 0-50, 2-40, 5-35, 25-40, 5-20, 1-20, or 2-15.

Particularly, X may be 0-90, Y may be 0-95, and Z may be 0-50. More particularly, X may be 1-85, Y may be 10-90, and Z may be 2-40. Still more particularly, X may be 2-80, Y may be 15-85, and Z may be 5-35.

In another embodiment, X may be 65-90, Y may be 5-30, and Z may be 1-20. In still another embodiment, X may be 70-85, Y may be 10-25, and Z may be 2-15.

In another embodiment, X may be 1-15, Y may be 75-90, and Z may be 5-20.

In another embodiment, X may be 5-20, Y may be 50-65, and Z may be 25-40.

Each active ingredient, i.e. PHC having each length of the alkyl chain, may be a single kind of PHC species, or may be a combination of two or more kinds of PHC species. The single kind of PHC species or each of the two or more kinds of PHC species constituting each active ingredient is not particularly limited so long as it is a PHC species having the corresponding length of the alkyl chain. The term corresponding length” means the length corresponding to each active ingredient. When each active ingredient is a combination of two or more kinds of PHC species, the term “amount of each active ingredient” refers to the total amount of the combination.

Each active ingredient, i.e. PHC having each length of the alkyl chain, may include, for example, any of the PHC species exemplified above and having the corresponding length of the alkyl chain. That is, each active ingredient may have any one or more features of PHC described above, provided that it has the corresponding length of the alkyl chain. Each active ingredient may also include, specifically, for example, a PHC species having the corresponding length of the alkyl chain, of which the acyl chain has no substitution group. Each active ingredient may also include, specifically, for example, a PHC species having the corresponding length of the alkyl chain, of which the alkyl chain has hydroxyl groups at C1, C3, and C4 and has no additional hydroxyl group other than these hydroxyl groups. Each active ingredient may also include, specifically, for example, a PHC species having the corresponding length of the alkyl chain, of which the alkyl chain has no or only one unsaturated double bond. The phrase “an active ingredient (i.e. PHC having a certain length of the alkyl chain) includes a certain PHC species” means that at least the certain PHC species is used as this active ingredient, and includes cases where this active ingredient is the certain PHC species and cases where this active ingredient is a combination of the certain PHC species and one or more kinds of other PHC species having the corresponding length of the alkyl chain. In cases of “an active ingredient (i.e. PHC having a certain length of the alkyl chain) includes a certain PHC species”, the ratio of the amount of the certain PHC species to the total amount of this active ingredient may be, for example, 30% or more, 40% or more, 50% or more, 60% or more, 70% or more, 80% or more, 90% or more, 95% or more, 97% or more, 99% or more, 99.5% or more, 99.9% or more, or 100%, by weight. Each active ingredient may have an acyl chain having a length selected from C14 to C26, e.g. from C14, C16, C18, C20, C22, C24, and C26. The phase “an active ingredient (i.e. PHC having a certain length of the alkyl chain) has an acyl chain having a length selected from C14 to C26” means that, when this active ingredient is a combination of two or more kinds of PHC species, each of the PHC species independently has an acyl chain having a length selected from C14 to C26, e.g. from C14, C16, C18, C20, C22, C24, and C26.

The PHC (A) may be a single kind of C16 PHS ceramide, or may be a combination of two or more kinds of C16 PHS ceramides. When the PHC (A) is a combination of two or more kinds of C16 PHS ceramides, X represents the total amount of the combination. The PHC (A) may include C16:0 PHS ceramide. The phase “the PHC (A) includes C16:0 PHS ceramide” means that at least C16:0 PHS ceramide is used as the PHC (A), and may include cases where the PHC (A) is C16:0 PHS ceramide or is a combination of C16:0 PHS ceramide and one or more kinds of other C16 PHS ceramides. The ratio of the amount of C16:0 PHS ceramide to the total amount of the PHC (A) may be, for example, 30% or more, 40% or more, 50% or more, 60% or more, 70% or more, 80% or more, 90% or more, 95% or more, 97% or more, 99% or more, 99.5% or more, 99.9% or more, or 100%, by weight. The PHC (A) may have any feature of PHC described above, provided that PHC (A) has a C16 PHS moiety. For example, the PHC (A) may have an acyl chain having a length selected from C14 to C26, e.g. from C14, C16, C18, C20, C22, C24, and C26. The phase “the PHC (A) has an acyl chain having a length selected from C14 to C26” means that, when the PHC (A) is a combination of two or more kinds of C16 PHS ceramides, each of the C16 PHS ceramides independently has an acyl chain having a length of from C14 to C26, e.g. from C14, C16, C18, C20, C22, C24, and C26.

The PHC (B) may be a single kind of C18 PHS ceramide, or may be a combination of two or more kinds of C18 PHS ceramides. When the PHC (B) is a combination of two or more kinds of C18 PHS ceramides, Y represents the total amount of the combination. The PHC (B) may include C18:0 PHS ceramide. The phase “the PHC (B) includes C18:0 PHS ceramide” means that at least C18:0 PHS ceramide is used as the PHC (B), and may include cases where the PHC (B) is C18:0 PHS ceramide or is a combination of C18:0 PHS ceramide and one or more kinds of other C18 PHS ceramides. The ratio of the amount of C18:0 PHS ceramide to the total amount of the PHC (B) may be, for example, 30% or more, 40% or more, 50% or more, 60% or more, 70% or more, 80% or more, 90% or more, 95% or more, 97% or more, 99% or more, 99.5% or more, 99.9% or more, or 100%, by weight. The PHC (B) may have any feature of PHC described above, provided that PHC (B) has a C18 PHS moiety. For example, the PHC (B) may have an acyl chain having a length of from C14 to C26, e.g. from C14, C16, C18, C20, C22, C24, and C26. The phase “the PHC (B) has an acyl chain having a length selected from C14 to C26” means that, when the PHC (B) is a combination of two or more kinds of C18 PHS ceramides, each of the C18 PHS ceramides independently has an acyl chain having a length of from C14 to C26, e.g. from C14, C16, C18, C20, C22, C24, and C26.

The PHC (C) may be a single kind of C20 PHS ceramide, or may be a combination of two or more kinds of C20 PHS ceramides. When the PHC (C) is a combination of two or more kinds of C20 PHS ceramides, Z represents the total amount of the combination. The PHC (C) may include C20:0 PHS ceramide. The phase “the PHC (C) includes C20:0 PHS ceramide” means that at least C20:0 PHS ceramide is used as the PHC (C), and may include cases where the PHC (C) is C20:0 PHS ceramide or is a combination of C20:0 PHS ceramide and one or more kinds of other C20 PHS ceramides. The ratio of the amount of C20:0 PHS ceramide to the total amount of the PHC (C) may be, for example, 30% or more, 40% or more, 50% or more, 60% or more, 70% or more, 80% or more, 90% or more, 95% or more, 97% or more, 99% or more, 99.5% or more, 99.9% or more, or 100%, by weight. The PHC (C) may have any feature of PHC described above, provided that PHC (C) has a C20 PHS moiety. For example, the PHC (C) may have an acyl chain having a length of from C14 to C26, e.g. from C14, C16, C18, C20, C22, C24, and C26. The phase “the PHC (C) has an acyl chain having a length selected from C14 to C26” means that, when the PHC (C) is a combination of two or more kinds of C20 PHS ceramides, each of the C20 PHS ceramides independently has an acyl chain having a length of from C14 to C26, e.g. from C14, C16, C18, C20, C22, C24, and C26.

As each of the active ingredients, a commercially available PHC may be used, or PHC appropriately prepared and obtained may be used. Methods for producing PHC are not particularly limited. PHC can be produced by, for example, known methods. PHC can be produced by, for example, a chemical synthesis method, enzymatic method, bioconversion method, fermentation method, extraction method, or a combination of these. PHC can be produced by, specifically, for example, a fermentation method using yeast. Alternatively, PHC can be produced by, specifically, for example, conversion of PHS. Methods for producing PHC can be independently chosen for each of the active ingredients.

Methods for producing PHS are not particularly limited. PHS can be produced by, for example, known methods. PHS can be produced by, for example, a chemical synthesis method, enzymatic method, bioconversion method, fermentation method, extraction method, or a combination of these. PHS can be produced by, specifically, for example, a fermentation method using yeast.

That is, each of the active ingredients may be, for example, PHC produced by a fermentation method, such as a fermentation method using yeast. Alternatively, each of the active ingredients may be, for example, PHC having a PHS moiety produced by a fermentation method, such as a fermentation method using yeast.

Examples of methods for producing PHC or PHS by a fermentation method using yeast, and producing PHC by conversion of PHS are described later.

Each of the PHCs constituting the active ingredients may be individually produced, or two or more of the PHCs constituting the active ingredients may be collectively produced as a mixture. A mixture containing two or more kinds of PHC species is also referred to as “PHC mixture”. Thus-produced PHC(s) and/or PHC mixture(s) may be appropriately combined and used as the active ingredients. That is, for example, two or more kinds of PHCs may be mutually combined, one or more kinds of PHC(s) and one or more kinds of PHC mixture(s) may be mutually combined, or two or more PHC mixtures may be mutually combined, to constitute the combination of the active ingredients. Alternatively, a PHC mixture may be used as the active ingredients as it is.

As each of the active ingredients, PHC purified to a desired extent may be used, or a material containing PHC may be used.

The composition may include only the active ingredients, or may include an ingredient other than the active ingredients. That is, the active ingredients may be present in the composition as it is, or may be present in the composition in combination with another ingredient. As the ingredient other than the active ingredients, a single kind of ingredient may be used, or two or more kinds of ingredients may be used in combination.

The ingredient other than the active ingredients is not particularly limited, so long as an intended effect is obtained. As the ingredient other than the active ingredients, ingredients acceptable depending on the intended purpose of the composition. Examples of the ingredient other than the active ingredients include physiologically acceptable ingredients. Examples of the physiologically acceptable ingredients include ingredients for a cosmetic or pharmaceutical, such as those generally used for cosmetic use or pharmaceutical use. The term “ingredient for a cosmetic” refers to an ingredient usable as an ingredient in a cosmetic. The term “ingredient for a pharmaceutical” refers to an ingredient usable as an ingredient in a pharmaceutical. Examples of such ingredients include excipients, binders, disintegrants, lubricants, stabilizers, diluents, surfactants, pH adjusters, vitamins, minerals, perfumes, pigments, preservatives, terpenoids, lipids, fatty acids, alcohols, and water. Particular examples of the ingredient other than the active ingredients include terpenoids, lipids, and fatty acids. Examples of the terpenoids include sterols, squalene, and pristane. Examples of the sterols include cholesterol and cholesterol sulfate. Examples of the lipids include triacylglycerols and glycerophospholipids. Examples of the triacylglycerols include triolein. Examples of the glycerophospholipids include phosphatidylethanolamine. Examples of the fatty acids include lauric acid (12:0), myristic acid (14:0), palmitic acid (16:0), stearic acid (18:0), arachidic acid (20:0), behenic acid (22:0), lignoceric acid (24:0), cerotic acid (26:0), myristoleic acid (14:1), palmitoleic acid (16:1), oleic acid (18:1), linoleic acid (18:2), and linolenic acid (18:3). Particular examples of the fatty acids include lauric acid, myristic acid, palmitic acid, stearic acid, arachidic acid, behenic acid, lignoceric acid, oleic acid, and linolenic acid. More particular examples of the fatty acids include lauric acid, palmitic acid, stearic acid, arachidic acid, behenic acid, and lignoceric acid. More particular examples of the fatty acids include lauric acid. Examples of such ingredients also include various physiologically active substances, such as antioxidants, anti-inflammatories, moisturizing ingredients, anti-ageing ingredients, anti-cellulite ingredients, skin whitening ingredients, skin tanning ingredients, and UV filters. Examples of such ingredients also include ceramides such as PHCs not selected as the active ingredients. As the ingredient other than the active ingredients, for example, one or more ingredients exemplified above may be at least selected. In other words, the ingredient other than the active ingredients may include one or more ingredients exemplified above. The phrase “a certain ingredient is selected as the ingredient other than the active ingredients” or “the ingredient other than the active ingredients includes a certain ingredient” means that at least the certain ingredient is used as the ingredient other than the active ingredients, and includes when the ingredient other than the active ingredients includes only the certain ingredient and when the ingredient other than the active ingredients includes only a combination of the certain ingredient and one or more kinds of other ingredients. All such ingredients as exemplified above can be used as, for example, ingredients for both a cosmetic and a pharmaceutical.

When the ingredient other than the active ingredients can form a salt, the ingredient other than the active ingredients may be a free compound, a salt thereof, or a combination thereof. For example, examples of salts for acidic groups such as carboxyl group, e.g. salts of fatty acids, include ammonium salt, salts with alkaline metal such as sodium and potassium, salts with alkaline earth metal such as calcium and magnesium, and salts with other metals such as aluminum and zinc.

The form of the composition is not particularly limited, so long as an intended effect is obtained. The form of the composition may be set according to various conditions such as the types of the active ingredients, the type(s) of the other ingredient(s), the amounts of the ingredients, the purpose of the, and the mode of administration of the composition. The composition may be formulated to an intended form. The composition may be provided, for example, in the form of any cosmetic or pharmaceutical composition applicable to skin. Examples of the cosmetic or pharmaceutical composition, especially examples of the cosmetic composition, include skin care cosmetics, makeup cosmetics, point makeup cosmetics, hair care products, hair setting agents, hair coloring agents, decolorizing agents, permanent agents, wave agents, body care cosmetics, UV care cosmetics. Examples of the skin care cosmetics include cleansing cosmetics (such as face washes and makeup removers), lotions, serums, packs, massage creams, milky lotions, creams, and essences. Examples of the makeup cosmetics include foundations, concealers, face powders, and make-up bases. Examples of the point makeup cosmetics include lipsticks, blushers, and eye makeups, such as eyeshadows, eyeliners, mascaras, and eyebrows. Examples of the hair care products include shampoos and conditioners, also referred to as rinse or treatment. A hair care product may be a product for, for example, obtaining such an effect as exemplified above in head hair and/or head skin. Examples of the hair setting agents include hair creams, hair waxes, hair liquids, and hair sprays. Examples of the hair coloring agents or decolorizing agents include hair manicures and hair colors. Examples of the body care cosmetics include body creams, soaps, body soaps, hand soaps, and antiperspirants. Examples of the UV care cosmetics include sunscreen cosmetics, suntan cosmetics, self-tanning cosmetics, and after-sun cosmetics. Examples of the cosmetic or pharmaceutical composition, especially examples of the pharmaceutical composition, include external preparations, such as external skin preparations. Examples of the external preparations include solid agents, such as powders, liquid agents, such as lotions and liniments, sprays, ointments, creams, gels, and patches, such as tapes and poultices. The composition may be provided in a form applicable to skin as it is, or may be prepared in a form applicable to skin before use.

The amounts of ingredients (namely, the active ingredients and optionally other ingredient(s)) in the composition are not particularly limited, so long as an intended effect is obtained. The amounts of ingredients in the composition can be appropriately set according to various conditions such as the types of the active ingredients, the type(s) of the other ingredient(s), the form of the composition, the purpose of the composition, and the mode of administration of the composition.

The total amount of the active ingredients present in the composition, for example, may be 0.01% or more, 0.1% or more, 1% or more, 5% or more, or 10% or more, by weight, may be 100% or less, 99.9% or less, 70% or less, 50% or less, 30% or less, 10% or less, 5% or less, or 1% or less, by weight, or may be within a range defined as a non-contradictory combination thereof. The total amount of the active ingredients present in the composition may specifically be, for example, 0.01-1%, 1-5%, 5-10%, 10-20%, 20-30%, or 30-50%, by weight. The total amount of the active ingredients present in the composition may specifically be, for example, 0.01-50%, 0.01-30%, 0.01-10%, or 0.01-5%, by weight. The total amount of the active ingredients present in the composition may specifically be, for example, 1-50%, 1-30%, 1-10%, or 1-5%, by weight. The total amount of the active ingredients present in the composition may specifically be, for example, 5-50%, 5-30%, or 5-10%, by weight.

When the composition contains ceramide(s) other than the active ingredients, the ratio of the total amount of the active ingredients to the total amount of ceramides in the composition may be, for example, 30% or more, 40% or more, 50% or more, 60% or more, 70% or more, 80% or more, 90% or more, 95% or more, 97% or more, 99% or more, 99.5% or more, or 99.9% or more, by weight.

Furthermore, the total amount of the active ingredients in the composition may be, for example, such an amount that the total application amount of the active ingredient is within a desired range when applying the composition to a body surface, such as skin or body hair.

Incidentally, the amount of each active ingredient when using a material containing the active ingredient should be calculated on the basis of the amount of the active ingredient per se in the material.

<2> Method

The method as described herein is a method including a step of applying the composition to an organism.

By the method as described herein, specifically by applying the composition to an organism, the condition of the application target of the composition may be improved, i.e. an effect of improving condition of the application target of the composition may be obtained. Hence, the method may improve the condition of the application target of the composition.

Improvement in condition is as described above. That is, the method may improve the condition of a body surface, such as skin and body hair. The method may be, particularly, a method for improving skin condition. The method may be a method for improving skin lamellar structure, improving skin barrier function, or improving skin moisturizing capacity. A method for improving skin barrier function or improving skin moisturizing capacity may be an embodiment of a method for improving skin lamellar structure. A method for improving skin moisturizing capacity may be an embodiment of a method for improving skin barrier function.

By the method, specifically by applying the composition to an organism, an effect based on improvement in condition of the application target of the composition may be obtained. Hence, the method may be a method for obtaining an effect based on improvement in condition of the application target of the composition.

The effect based on improvement in condition of the application target of the composition, i.e. the application target of the active ingredients is as described above. That is, the method may be a method for preventing and/or treating a symptom relating to deterioration in condition of the application target of the composition. The method may be, particularly, a method for preventing and/or treating a symptom relating to deterioration in skin condition.

The application scheme, e.g. application target, application timing, application period, application number, application amount, and other conditions relating to application, of the composition is not particularly limited, so long as an intended effect is obtained. The application scheme of the composition can be appropriately selected according to various conditions such as the types and amounts of the active ingredients, the type(s) and amount(s) of other ingredient(s), the type and form (dosage form) of the composition, and the type, age, and health condition of the application target, and the purpose of the composition.

The application target of the composition are as described above. That is, the composition can be applied to, for example, such an application target as exemplified above. The composition can be applied to, for example, such an application target as exemplified above. The composition can be applied to the organism by an appropriate means such as spreading, pasting, and spraying.

The application period of the composition may be, for example, 1 hour or longer, 6 hours or longer, 1 day or longer, 3 days or longer, 1 week or longer, 4 weeks or longer, 2 months or longer, 6 months or longer, 12 months or longer, 2 years or longer, 5 years or longer, or 10 years or longer. The composition may be applied, for example, routinely or only during a specific period. The composition may be applied, for example, continuously or intermittently. The composition may be applied, for example, until an intended effect is obtained. The composition may be applied, for example, once a day, or two or more times a day as divided portions. The composition may be applied, for example, every day, or once in several days. The application amount of the composition at the time of each application may or may not be constant in terms of the application amount of the active ingredients. The application amount of the composition at the time of each application in terms of the application amount of the active ingredients, for example, may be 0.1 μg/cm² or more, 0.5 μg/cm² or more, 1 μg/cm² or more, 5 μg/cm² or more, or 10 μg/cm² or more, may be 500 mg/cm² or less, 100 mg/cm² or less, 50 mg/cm² or less, 10 mg/cm² or less, 5 mg/cm² or less, 1 mg/cm² or less, 0.5 mg/cm² or less, or 0.1 mg/cm² or less, or may be within a range defined as a combination thereof. The application amount of the composition at the time of each application in terms of the application amount of the active ingredients may specifically be, for example, 0.1 μg/cm²-500 mg/cm².

<3> Method for Producing PHC

Hereinafter, examples of methods for producing PHC or PHS by a fermentation method using yeast, and producing PHC by conversion of PHS are described. The product of the fermentation method, i.e. PHS and/or PHC, is also referred to as “objective substance”. The yeast used in the fermentation method is also referred to as “the yeast of the present invention”.

<3-1> Yeast

The yeast as described herein is yeast having an ability to produce an objective substance. The “ability to produce an objective substance” may also be referred to as “objective substance-producing ability”.

The term “yeast having an objective substance-producing ability” refers to yeast that is able to produce and accumulate an objective substance in a culture medium or cells of the yeast to such a degree that the objective substance can be collected, when the yeast is cultivated in the culture medium. The culture medium may be a culture medium that can be used in the fermentation method, and may specifically be a culture medium containing a fatty acid. The yeast having an objective substance-producing ability may also be yeast that is able to produce and accumulate an objective substance in a culture medium or cells of the yeast in an amount larger than that obtainable with a non-modified strain. The term “non-modified strain” may refer to a reference strain that has not been modified so that an objective substance-producing ability is imparted or enhanced. Examples of the non-modified strain include a wild-type strain and parent strain, such as Saccharomyces cerevisiae strains BY4742 (ATCC 201389; EUROSCARF Y10000), S288C (ATCC 26108), and NCYC 3608. The yeast having an objective substance-producing ability may also be yeast that is able to produce and accumulate an objective substance in a culture medium in an amount of 5 mg/L or more, or 10 mg/L or more.

The yeast is not particularly limited so long as it can be used for the fermentation method. The yeast may be budding yeast, or may be fission yeast. The yeast may be haploid, diploid, or more polyploid.

Examples of the yeast include yeast belonging to the genus Saccharomyces such as Saccharomyces cerevisiae, the genus Pichia (also referred to as the genus Wickerhamomyces) such as Pichia ciferrii, Pichia sydowiorum, and Pichia pastoris, the genus Candida such as Candida utilis, the genus Hansenula such as Hansenula polymorpha, the genus Schizosaccharomyces such as Schizosaccharomyces pombe. Some species of the genus Pichia has been reclassified into the genus Wickerhamomyces (Int J Syst Evol Microbiol. 2014 March; 64(Pt 3):1057-61). Therefore, for example, Pichia ciferrii and Pichia sydowiorum are also called Wickerhamomyces ciferrii and Wickerhamomyces sydowiorum, respectively. The term “Pichia” should include such species that had been classified into the genus Pichia but have been reclassified into another genus such as Wickerhamomyces.

Specific examples of Saccharomyces cerevisiae include strains BY4742 (ATCC 201389; EUROSCARF Y10000), S288C (ATCC 26108), Y006 (FERM BP-11299), NCYC 3608, and derivative strains thereof. Specific examples of Pichia ciferrii (Wickerhamomyces ciferrii) include strain NRRL Y-1031 (ATCC 14091), strain CS.PCΔPro2 (Schorsch et al., 2009, Curr Genet. 55, 381-9.), strains disclosed in WO 95/12683, and derivative strains thereof. Specific examples of Pichia sydowiorum (Wickerhamomyces sydowiorum) include strain NRRL Y-7130 (ATCC 58369) and derivative strains thereof.

These strains are available from, for example, the American Type Culture Collection (ATCC, Address: P.O. Box 1549, Manassas, VA 20108, United States of America), EUROpean Saccharomyces Cerevisiae ARchive for Functional Analysis (EUROSCARF, Address: Institute for Molecular Biosciences, Johann Wolfgang Goethe-University Frankfurt, Max-von-Laue Str. 9; Building N250, D-60438 Frankfurt, Germany), the National Collection of Yeast Cultures (NCYC, Address: Institute of Food Research, Norwich Research Park, Norwich, NR4 7UA, UK), or depositary institutions corresponding to deposited strains. That is, for example, in cases of ATCC strains, registration numbers are assigned to the respective strains, and the strains can be ordered using these registration numbers (refer to www.atcc.org). The registration numbers of the strains are listed in the catalogue of the American Type Culture Collection (ATCC).

The yeast may be yeast inherently having an objective substance-producing ability, or may be yeast modified so that it has an objective substance-producing ability. The yeast having an objective substance-producing ability can be obtained by imparting an objective substance-producing ability to yeast such as those mentioned above, or by enhancing an objective substance-producing ability of yeast such as those mentioned above.

Hereafter, methods for imparting or enhancing an objective substance-producing ability will be specifically exemplified. All the modifications for imparting or enhancing an objective substance-producing ability may be used independently or in any appropriate combination. Modifications for constructing the yeast can be performed in an arbitrary order.

An objective substance-producing ability may be imparted or enhanced by modifying yeast so that the expression and/or activity of one or more kinds of proteins involved in production of the objective substance are increased or reduced. That is, the yeast may have been modified so that the expression and/or activity of one or more kinds of proteins involved in production of the objective substance are increased or reduced. The term “protein” also includes so-called peptides such as polypeptides. Examples of the proteins involved in production of the objective substance include enzymes that catalyze the synthesis of the objective substance, also referred to as “biosynthetic enzyme of objective substance”, enzymes that catalyze a reaction branching away from the biosynthetic pathway of the objective substance to generate a compound other than the objective substance (also referred to as “biosynthetic enzyme of byproduct”), enzymes that catalyze decomposition of the objective substance (also referred to as “decomposition enzyme of objective substance”), proteins that affect, e.g. increase or reduce, the activity of an enzyme such as those described above.

The protein of which the expression and/or activity is to be increased or reduced can be appropriately chosen depending on the type of the objective substance and on the types and activities of the proteins involved in production of the objective substance and inherently possessed by the yeast. For example, the expression and/or activity of one or more kinds of proteins selected from biosynthetic enzymes of the objective substance may be increased. Also, for example, the expression and/or activity of one or more kinds of proteins selected from biosynthetic enzymes of a byproduct and decomposition enzymes of the objective substance may be reduced.

The activity of a protein can be increased by, for example, increasing the expression of a gene encoding the protein. The expression and/or activity of a protein may be increased by, for example, increasing the copy number of a gene encoding the protein, or modifying an expression control sequence of a gene encoding the protein. The activity of a protein can be reduced by, for example, reducing the expression of a gene encoding the protein or disrupting a gene encoding the protein. The expression and/or activity of a protein can be reduced by, for example, deletion of a gene encoding the protein. When increasing or reducing the expression and/or activity of two or more kinds of proteins, methods for increasing or reducing the expression and/or activity of each of the proteins can be independently chosen. The expression of a gene is also referred to as “the expression of a protein, i.e. the protein encoded by the gene”. Such methods of increasing or reducing the expression and/or activity of a protein are well known in the art.

Specific examples of the proteins involved in production of the objective substance include proteins encoded by the LCB1, LCB2, TSC10, SUR2, LAG1, LAC1, LIP1, SER1, SER2, SER3, YPC1, NEM1, SPO7, LCB4, LCB5, ELO3, CKA2, ORM2, and CHA1 genes.

The yeast may have been modified so that the expression and/or activity of one or more of proteins encoded by the LCB1, LCB2, TSC10, SUR2, LAG1, LAC1, LIP1, SER1, SER2, SER3, and YPC1 genes is increased, and/or that the expression and/or activity of one or more of proteins encoded by the YPC1, NEM1, SPO7, LCB4, LCB5, ELO3, CKA2, ORM2, and CHA/genes is reduced. The expression and/or activity of Ypc1p may be increased, for example, in cases of producing PHS. Alternatively, the expression and/or activity of Ypc1p may be reduced, for example, in cases of producing PHC.

Examples of these genes and proteins include those of yeast such as S. cerevisiae and Pichia ciferrii.

The genes and proteins used for breeding the yeast may have, for example, the nucleotide sequences and amino acid sequences of known genes and proteins, such as those exemplified above, respectively. Also, the genes and proteins used for breeding L-amino acid-producing bacteria may be variants of known genes and proteins, such as those exemplified above, respectively, so long as the original function (such as activity and property) thereof is maintained.

<3-2> Method for Producing PHC

The fermentation method can be carried out by cultivating the yeast in a culture medium. In the fermentation method, a single kind of objective substance may be produced, or two or more kinds of objective substances may be produced.

The culture medium to be used is not particularly limited, so long as the yeast can proliferate in it, and an objective substance can be produced. As the culture medium, for example, a usual culture medium used for cultivating yeast can be used. Examples of such a culture medium include SD medium, SG medium, SDTE medium, and YPD medium. The culture medium may contain a carbon source, a nitrogen source, a phosphorus source, and a sulfur source, as well as components selected from other various organic components and inorganic components as required. The types and concentrations of the culture medium components can be appropriately determined according to various conditions such as the type of the yeast to be used and the type of the objective substance to be produced.

The culture medium may contain a fatty acid. Use of the fatty acid may result in an increased production of the objective substance. That is, production of the objective substance by the yeast may be increased in the presence of the fatty acid as compared with in the absence of the fatty acid. Examples of the increased production of the objective substance include an increased production amount of the objective substance, an increased production rate of the objective substance, and an increased yield of the objective substance. In addition, use of the fatty acid may enable regulating the composition, such as the length, of the alkyl chain of the objective substance. Examples of such regulation include regulation of production of an objective substance including a specific alkyl chain, and regulation of ratio of the production amount of an objective substance including a specific alkyl chain to the total amount of all products. Such ratio is also referred to as “production ratio”. Examples of the specific alkyl chain include an alkyl chain having a specific length. The term “total amount of all products” may refer to, for example, the total amount of two or more kinds of PHS species, such as all produced PHS species, or the total amount of two or more kinds of PHC species, such as all produced PHC species.

That is, specifically, use of the fatty acid may result in an increased production of an objective substance including a specific alkyl chain depending on the kind of the fatty acid. For example, use of a fatty acid having a carbon number of n may result in an increased production of an objective substance including an alkyl chain having a carbon number of n+2. In other words, when the fatty acid has a carbon number of n, the objective substance may include a PHS or PHC species including an alkyl chain having a carbon number of n+2, and production of this PHS or PHC species may be increased due to the presence of the fatty acid. The phase “the objective substance includes a PHS or PHC species” means that at least this PHS or PHC species is produced as the objective substance, and may include cases where the objective substance includes this PHS and/or PHC species or includes a mixture containing this PHS and/or PHC species.

Also, specifically, use of the fatty acid may result in an increased ratio of the production amount of an objective substance including a specific alkyl chain to the total amount of products depending on the kind of the fatty acid. For example, use of a fatty acid having a carbon number of n may result in an increased ratio of the production amount of an objective substance including an alkyl chain having a carbon number of n+2 to the total amount of products. In other words, when the fatty acid has a carbon number of n, the objective substance may include a PHS or PHC species including an alkyl chain having a carbon number of n+2, and the ratio of the production amount of this PHS or PHC species to the total amount of products may be increased due to the presence of the fatty acid.

The length and the unsaturation degree of the fatty acid may vary. The fatty acid may have a length of, for example, C12 to C24, such as C12, C14, C16, C18, C20, C22, and C24. The fatty acid may have a length of, for example, particularly, C14, C16, or C18. The length of the fatty acid may be interpreted as the carbon number (i.e. the number of carbon atoms) of the fatty acid. The fatty acid may be saturated, or may be unsaturated. The fatty acid may have one or more unsaturated double bonds. Specific examples of the fatty acid include lauric acid (12:0), myristic acid (14:0), palmitic acid (16:0), stearic acid (18:0), arachidic acid (20:0), behenic acid (22:0), lignoceric acid (24:0), myristoleic acid (14:1), palmitoleic acid (16:1), oleic acid (18:1), linoleic acid (18:2), and linolenic acid (18:3). Particular examples of the fatty acid include myristic acid (14:0), palmitic acid (16:0), and stearic acid (18:0). More particular examples of the fatty acid include myristic acid (14:0). Use of myristic acid (14:0) may result in an increased production or production ratio of C16 PHS or PHC, such as C16:0 PHS or PHC. Use of palmitic acid (16:0) may result in an increased production or production ratio of C18 PHS or PHC, such as C18:0 PHS or PHC. Use of stearic acid (18:0) may result in an increased production or production ratio of C20 PHS or PHC, such as C20:0 PHS or PHC. As the fatty acid, a single kind of fatty acid may be used, or two or more kinds of fatty acids may be used in combination.

The fatty acid may be used as a free compound, a salt thereof, or a mixture thereof. That is, the term “fatty acid” may refer to a fatty acid in a free form, a salt thereof, or a mixture thereof, unless otherwise stated. Examples of the salt can include, for example, ammonium salt, sodium salt, and potassium salt. As the salt of the precursor, a single kind of salt may be employed, or two or more kinds of salts may be employed in combination.

The fatty acid may be present in the culture medium over the whole period of the culture, or may be present in the culture medium during only a partial period of the culture. That is, the phrase “cultivating yeast in a culture medium containing a fatty acid” does not necessarily mean that the fatty acid is present in the culture medium over the whole period of the culture. For example, the fatty acid may be or may not be contained in the culture medium from the start of the culture. When the fatty acid is not contained in the culture medium at the time of the start of the culture, the fatty acid is supplied to the culture medium after the start of the culture. Timing of the supply can be appropriately determined according to various conditions such as the length of culture period. For example, the fatty acid may be supplied to the culture medium after the yeast fully grows. Furthermore, in any case, the fatty acid may be additionally supplied to the culture medium as required. Means for supplying the fatty acid to the culture medium is not particularly limited. For example, the fatty acid can be supplied to the culture medium by feeding a feed medium containing the fatty acid to the culture medium. The concentration of the fatty acid in the culture medium is not particularly limited so long as the objective substance can be produced. For example, the concentration of the fatty acid in the culture medium may be 0.1 g/L or higher, 1 g/L or higher, 2 g/L or higher, 5 g/L or higher, or 10 g/L or higher, may be 200 g/L or lower, 100 g/L or lower, 50 g/L or lower, or 20 g/L or lower, or may be within a range defined with a combination thereof. The concentration of the fatty acid in the culture medium may be, for example, 0.1 g/L to 200 g/L, 1 g/L to 100 g/L, or 5 g/L to 50 g/L. The fatty acid may be or may not be present in the culture medium at a concentration within the range exemplified above during the whole period of the culture. For example, the fatty acid may be present in the culture medium at a concentration within the range exemplified above at the start of the culture, or it may be added to the culture medium so that a concentration within the range exemplified above is attained after the start of the culture.

The culture medium may contain an additive that is able to associate with, bind to, solubilize, and/or capture the objective substance (WO2017/033463). Use of the additive may result in an increased production of the objective substance. That is, the amount produced of the objective substance by the yeast may be increased in the presence of the additive as compared with in the absence of the additive. Use of the additive may specifically result in an increased production of the objective substance in the culture medium. The production of the objective substance in the culture medium may also be referred to as “excretion of the objective substance”. The expression “associating with, binding to, solubilizing, and/or capturing an objective substance” may specifically mean increasing the solubility of the objective substance into the culture medium. Examples of the additive include cyclodextrins and zeolites. The number of glucose residues constituting cyclodextrins is not particularly limited, and it may be, for example, 5, 6, 7, or 8. That is, examples of cyclodextrins include cyclodextrin consisting of 5 glucose residues, alpha-cyclodextrin, beta-cyclodextrin, gamma-cyclodextrin, and derivatives thereof. Examples of cyclodextrin derivatives include cyclodextrins into which one or more functional groups have been introduced. The type, number, and amount of the functional group, and the position to which the functional group is introduced are not particularly limited as long as the derivative is able to associate with, bind to, solubilize, and/or capture the objective substance. The functional group may be introduced to, for example, hydroxyl group of C2, C3, C6, or a combination thereof, which may result in an increased solubility of cyclodextrin itself. Examples of the functional group include alkyl groups and hydroxyalkyl groups. The alkyl groups and hydroxyalkyl groups each may have a linear alkyl chain or may have a branched alkyl chain. The alkyl groups and hydroxyalkyl groups each may have a carbon number of, for example, 1, 2, 3, 4, or 5. Specific examples of the alkyl groups include methyl, ethyl, propyl, butyl, pentyl, isopropyl, and isobutyl groups. Specific examples of the hydroxyalkyl groups include hydroxymethyl, hydroxyethyl, hydroxypropyl, hydroxybutyl, hydroxypentyl, hydroxyisopropyl, and hydroxyisobutyl groups. Specific examples of cyclodextrin derivatives include methyl-alpha-cyclodextrin, methyl-beta-cyclodextrin, hydroxypropyl-alpha-cyclodextrin such as 2-hydroxypropyl-alpha-cyclodextrin, and hydroxypropyl-beta-cyclodextrin such as 2-hydroxypropyl-beta-cyclodextrin. The types of zeolites are not particularly limited. As the additive, a single kind of additive may be used, or two or more kinds of additives may be used in combination.

The additive may be contained in the culture medium over the whole period of the culture, or may be contained in the culture medium during only a partial period of the culture. That is, the phrase “cultivating yeast in a culture medium containing an additive” does not necessarily mean that the additive is present in the culture medium over the whole period of the culture. For example, the additive may be or may not be present in the culture medium from the start of the culture. When the additive is not present in the culture medium at the time of the start of the culture, the additive is supplied to the culture medium after the start of the culture. Timing of the supply can be appropriately determined according to various conditions such as the length of culture period. For example, the additive may be supplied to the culture medium after the yeast fully grows. Furthermore, in any case, the additive may be additionally supplied to the culture medium as required. Means for supplying the additive to the culture medium is not particularly limited. For example, the additive can be supplied to the culture medium by feeding a feed medium containing the additive to the culture medium. The concentration of the additive in the culture medium is not particularly limited so long as the objective substance can be produced. For example, the concentration of the additive in the culture medium may be 0.1 g/L or higher, 1 g/L or higher, 2 g/L or higher, 5 g/L or higher, or 10 g/L or higher, may be 200 g/L or lower, 100 g/L or lower, 50 g/L or lower, or 20 g/L or lower, or may be within a range defined with a combination thereof. The concentration of the additive in the culture medium may be, for example, 0.1 g/L to 200 g/L, 1 g/L to 100 g/L, or 5 g/L to 50 g/L. The additive may be or may not be present in the culture medium at a concentration within the range exemplified above during the whole period of the culture. For example, the additive may be present in the culture medium at a concentration within the range exemplified above at the start of the culture, or it may be supplied to the culture medium so that a concentration within the range exemplified above is attained after the start of the culture.

Specific examples of the carbon source include, for example, saccharides such as glucose, fructose, sucrose, lactose, galactose, xylose, arabinose, blackstrap molasses, starch hydrolysates, and hydrolysates of biomass, organic acids such as acetic acid, fumaric acid, citric acid, and succinic acid, alcohols such as glycerol, crude glycerol, and ethanol, and fatty acids. That is, the fatty acid described above may also be used as the carbon source. The fatty acid described above may be or may not be used as the sole carbon source. However, usually, at least a carbon source other than the fatty acid described above may be used. As the carbon source, a single kind of carbon source may be used, or two or more kinds of carbon sources may be used in combination.

Specific examples of the nitrogen source include, for example, ammonium salts such as ammonium sulfate, ammonium chloride, and ammonium phosphate, organic nitrogen sources such as peptone, yeast extract, meat extract, and soybean protein decomposition products, ammonia, and urea. Ammonia gas or aqueous ammonia used for adjusting pH may also be used as the nitrogen source. As the nitrogen source, a single kind of nitrogen source may be used, or two or more kinds of nitrogen sources may be used in combination.

Specific examples of the phosphate source include, for example, phosphoric acid salts such as potassium dihydrogenphosphate and dipotassium hydrogenphosphate, and phosphoric acid polymers such as pyrophosphoric acid. As the phosphate source, a single kind of phosphate source may be used, or two or more kinds of phosphate sources may be used in combination.

Specific examples of the sulfur source include, for example, inorganic sulfur compounds such as sulfates, thiosulfates, and sulfites, and sulfur-containing amino acids such as cysteine, cystine, and glutathione. As the sulfur source, a single kind of sulfur source may be used, or two or more kinds of sulfur sources may be used in combination.

Specific examples of other various organic components and inorganic components include, for example, inorganic salts such as sodium chloride and potassium chloride; trace metals such as iron, manganese, magnesium, and calcium; vitamins such as vitamin B1, vitamin B2, vitamin B6, nicotinic acid, nicotinamide, and vitamin B12; amino acids; nucleic acids; and organic components including these such as peptone, casamino acid, yeast extract, and soybean protein decomposition product. As other various organic components and inorganic components, a single kind of component may be used, or two or more kinds of components may be used in combination.

Furthermore, when an auxotrophic mutant that requires an amino acid, a nucleic acid, or the like for growth thereof is used, it is preferable to supplement a required nutrient to the culture medium.

The culture conditions are not particularly limited so long as the can proliferate, and the objective substance can be produced. The culture can be performed, for example, under conditions typically used when cultivating yeast. The culture conditions can be appropriately determined according to various conditions such as the type of yeast to be used and the type of objective substance to be produced.

The culture can be performed by using a liquid medium under an aerobic condition, a microaerobic condition, or an anaerobic condition. The culture can be performed under an aerobic condition. The term “aerobic condition” may refer to a condition where the dissolved oxygen concentration in the liquid medium is 0.33 ppm or higher, or 1.5 ppm or higher. In cases of the aerobic condition, the oxygen concentration can be controlled to be, for example, 5 to 50%, or about 10 to 20%, of the saturated oxygen concentration. Specifically, the aerobic culture can be performed with aeration or shaking. The term “microaerobic condition” may refer to a condition where oxygen is supplied to the culture system but the dissolved oxygen concentration in the liquid medium is lower than 0.33 ppm. The term “anaerobic condition” may refer to a condition where oxygen is not supplied to the culture system. The culture temperature may be, for example, 25 to 35° C., 27 to 33° C., or 28 to 32° C. The pH of the culture medium may be, for example, 3 to 10, or 4 to 8. The pH of the culture medium may be adjusted as required during the culture. For adjusting pH, inorganic or organic acidic or alkaline substances, such as ammonia gas and so forth, can be used. The culture period may be, for example, 10 to 200 hours, or 15 to 120 hours. The culture condition may be held constant during the entire period of the culture, or may be altered during the culture. The culture can be performed as batch culture, fed-batch culture, continuous culture, or a combination of these. Furthermore, the culture may be performed as two steps of a seed culture and a main culture. In such a case, the culture conditions of the seed culture and the main culture may or may not be the same. For example, both the seed culture and the main culture may be performed as batch culture. Alternatively, for example, the seed culture may be performed as batch culture, and the main culture may be performed as fed-batch culture or continuous culture.

By culturing the yeast under such conditions, the objective substance can be accumulated in the culture medium and/or cells of the yeast.

Production of the objective substance can be confirmed by known methods used for detection or identification of compounds. Examples of such methods include, for example, HPLC, UPLC, LC/MS, GC/MS, and NMR. These methods may be used independently or in any appropriate combination.

The produced objective substance can be appropriately collected. The produced objective substance can be collected by known methods used for separation and purification of compounds. Examples of such methods include, for example, ion-exchange resin method, membrane treatment, precipitation, and crystallization. These methods may be used independently or in any appropriate combination. When the objective substance accumulates in cells, the cells can be disrupted with, for example, ultrasonic waves or the like, and then the objective substance can be collected from the supernatant obtained by removing the cells from the cell-disrupted suspension by centrifugation. The objective substance to be collected may be a free compound, a salt thereof, or a mixture thereof.

Furthermore, when the objective substance precipitates in the culture medium, it can be collected by centrifugation, filtration, or the like. The objective substance precipitated in the culture medium may also be isolated together with the objective substance dissolved in the culture medium after the objective substance dissolved in the culture medium is crystallized.

The collected objective substance may contain additional substance(s) such as yeast cells, culture medium components, moisture, and by-product metabolites of the yeast, in addition to the objective substance. The purity of the collected objective substance may be, for example, 30% (w/w) or higher, 50% (w/w) or higher, 70% (w/w) or higher, 80% (w/w) or higher, 90% (w/w) or higher, or 95% (w/w) or higher.

When PHS is produced by cultivation of the yeast, the thus-produced PHS can be converted to a corresponding PHC. Provided herein is a method for producing PHC, the method including the step of producing PHS by the fermentation method, and converting the PHS to the PHC.

PHS produced by cultivation of the yeast can be used for the conversion to PHC as it is, or after being subjected to an appropriate treatment such as concentration, dilution, drying, dissolution, fractionation, extraction, and purification, as required. That is, as PHS, for example, a product purified to a desired extent may be used, or a material containing PHS may be used. The material containing PHS is not particularly limited so long as the conversion of PHS to PHC proceeds. Specific examples of the material containing PHS include a culture broth containing PHS, a supernatant separated from the culture broth, and processed products thereof such as concentrated products, such as concentrated liquid, thereof and dried products thereof.

Methods for converting PHS to PHC are not particularly limited.

PHS can be converted to PHC by, for example, a chemical reaction with a fatty acid (J. Biol. Chem. July 2002 277 (29): 25847-5). The chemical reaction can be carried out under, for example, typical conditions for condensing an amine and a carboxylic acid to form an amide bond. Specifically, the chemical reaction can be carried out by, for example, using a condensation agent. Examples of the condensation agent include carbodiimides such as water soluble carbodiimides (WSCs). Specific examples of WSCs include 1-Ethyl-3-(3-dimethylaminopropyl)carbodiimide hydrochloride. The fatty acid is not particularly limited so long as it provides the acyl chain of the PHC to be produced. That is, examples of the fatty acid include those corresponding to the acyl chains of the PHCs exemplified above. Specific examples of the fatty acid include myristic acid (14:0), palmitic acid (16:0), stearic acid (18:0), arachidic acid (20:0), behenic acid (22:0), lignoceric acid (24:0), cerotic acid (26:0), myristoleic acid (14:1), palmitoleic acid (16:1), oleic acid (18:1), linoleic acid (18:2), and linolenic acid (18:3). Particular examples of the fatty acid include stearic acid (18:0). As PHS, a single kind of PHS species may be used, or two or more kinds of PHS species may be used in combination. As the fatty acid, a single kind of fatty acid may be used, or two or more kinds of fatty acids may be used in combination. Use of two or more kinds of PHS species and/or two or more kinds of fatty acids may result in production of a mixture of two or more kinds of PHC species.

Confirmation of the production of PHC and collection of PHC can be carried out in the same manners as those for the fermentation method. The purity of PHC collected may be, for example, 30% (w/w) or higher, 50% (w/w) or higher, 70% (w/w) or higher, 80% (w/w) or higher, 90% (w/w) or higher, or 95% (w/w) or higher.

PHC produced as described above can be used as the active ingredient(s) as it is, or after being subjected to an appropriate treatment such as concentration, dilution, drying, dissolution, fractionation, extraction, and purification, as required. That is, as the active ingredient(s), for example, a product purified to a desired extent may be used, or a material containing PHC may be used. The material containing PHC is not particularly limited, so long as it is acceptable depending on the purpose of the composition. Specific examples of the material containing PHC include a culture broth or a conversion reaction mixture containing PHC, a supernatant separated from the culture broth or conversion reaction mixture, and processed products thereof such as concentrated products, such as concentrated liquid, thereof and dried products thereof.

<4> Use of the Active Ingredient

Use of the active ingredients for the purpose as exemplified above is described herein. That is, disclosed herein, for example, is the use of the active ingredients for improving the condition of the application target of the active ingredients, e.g. the application target of the composition, and the use of the active ingredients in manufacture of a composition, e.g. a cosmetic or pharmaceutical composition, for improving the condition of the application target thereof. Also disclosed herein, for example, is the use of the active ingredients for improving a property of a composition, e.g., a cosmetic or pharmaceutical composition.

Also disclosed herein are the active ingredients for use in the purpose as exemplified above. That is, disclosed herein, for example, is the active ingredients for use in improving condition of the application target of the active ingredients, e.g. the application portion of the composition, and the active ingredients for use in manufacture of a composition, e.g. a cosmetic or pharmaceutical composition, for improving condition of the application target thereof. Also disclosed, for example, are the active ingredients for use in improving a property of a composition, e.g. a cosmetic or pharmaceutical composition.

### EXAMPLES

The present invention will be more specifically explained with reference to the following non-limiting examples.

<1> Preparation of PHC

C18:0 PHS was obtained as a commercially available product from Tokyo Chemical Industry. C16:0 PHS and C20:0 PHS were obtained using contract manufacturing services of Tokyo Chemical Industry. Each of these PHS species was condensed with a fatty acid, specifically, palmitic acid (16:0), stearic acid (18:0), or behenic acid (22:0), using a condensation agent WSCI-HCl (1-Ethyl-3-(3-dimethylaminopropyl)carbodiimide hydrochloride, WATANABE CHEMICAL INDUSTRIES), to prepare a corresponding PHC species. In the Examples, PHC of Cn:m PHS and Cx:y fatty acid is also referred to as “Cx:y-PHSn:m”.

<2> Analysis of Property of Composition Containing PHC (1)

<2-1> Preparation of Compositions

PHC(s), cholesterol, and lauric acid in amounts shown in Table 1 were mixed. The mixture was heated at 95° C. to be melted, and then cooled to the room temperature, to prepare each sample.

<2-2> Measurement of Melting Point

Each sample was heated to 70° C., and the temperature of the sample was further raised at a rate of 1° C./5 min. The temperature at which the sample was completely melted was taken as the melting point of the sample.

<2-3> Measurement of Lamellar Structure-Forming Ability

Each sample was applied to a microscope slide, and a cover slip was placed thereon. The slide was located on a hotplate of 120° C. to melt the sample, and then was allowed to cool. The Maltese cross pattern of the sample was observed with a polarizing microscope. The lamellar structure-forming ability of the sample was evaluated and graded with scores ranging from 1 to 5 on the basis of observation of the Maltese cross pattern as follows.
Score 5: Maltese cross pattern is observed over the whole visual field.
Score 4: Maltese cross pattern is observed almost over the whole visual field.
Score 3: Maltese cross pattern is observed at a part of the visual field.
Score 2: Maltese cross pattern is slightly observed.
Score 1: Maltese cross pattern is not observed at all.

<2-4> Results

Results are shown in Table 1. The samples 1 and 2, each containing a combination of three kinds of PHC species having different lengths of alkyl chains, showed a lower melting point and a higher lamellar structure-forming ability than the samples 3-5, each containing a single kind of PHC species. In addition, the samples 1 and 2 showed a higher lamellar structure-forming ability than the sample 6 containing a combination of three kinds of PHC species having different lengths of acyl chains. That is, it was indicated that a combined use of multiple PHC species having different lengths of alkyl chains improves a property of a composition containing the same as compared with cases of using a single kind of PHC species solely or using multiple PHC species having different lengths of acyl chains.

TABLE 1
Melting point and lamellar structure-
forming ability of compositions

| Sample No. | | 1 | 2 | 3 | 4 | 5 | 6 |
|---|---|---|---|---|---|---|---|
| Amount | C18:0-PHS16:0 | 10 | 18 | 30 | | | |
| [mg] | C18:0-PHS18:0 | 10 | 6 | | 30 | | 10 |
| | C18:0-PHS20:0 | 10 | 6 | | | 30 | |
| | C16:0-PHS18:0 | | | | | | 10 |
| | C22:0-PHS18:0 | | | | | | 10 |
| | Cholesterol | 30 | 30 | 30 | 30 | 30 | 30 |
| | Lauric acid | 30 | 30 | 30 | 30 | 30 | 30 |

| | | | | | | |
|---|---|---|---|---|---|---|
| Total [mg] | 90 | 90 | 90 | 90 | 90 | 90 |
| Melting point [° C.] | 72 | 71 | 81 | 86 | 90 | 70 |
| Lamellar structure- | 5 | 5 | 3 | 3 | 2 | 3 |
| forming ability | | | | | | |

<3> Analysis of Property of Composition Containing PHC (2)

<3-1> Preparation of Compositions

PHCs in a mixing ratio shown in Table 2 were mixed in a mortar, to prepare each sample.

<3-2> Measurement of Melting Point

Each sample in an amount of approximately 2 mg was heated under a nitrogen atmosphere using a differential scanning calorimeter DSC7000X (Hitachi High-Tech Science Corporation) with the temperature raised from 25° C. at a rate of 5° C./min. The temperature at the intersection of the base line and the inflection point of endothermic peak was taken as the melting point of the sample.

<3-3> Results

Results are shown in Table 2. The samples 7-10, each having a combination of two or more kinds of PHC species having different lengths of alkyl chains, showed a melting point lower than, respectively, the samples 11 and 12, the samples 12 and 13, the samples 11 and 13, and the samples 11-13, each having a single kind of PHC species. That is, again, it was indicated that a combined use of multiple PHC species having different lengths of alkyl chains improves a property of a composition containing the same as compared with cases of using a single kind of PHC species solely.

TABLE 2
Melting point of compositions

| Sample No. | | 7 | 8 | 9 | 10 | 11 | 12 | 13 |
|---|---|---|---|---|---|---|---|---|
| Mixing | C18:0-PHS16:0 | 20 | | 20 | 20 | 100 | | |
| ratio | C18:0-PHS18:0 | 80 | 20 | | 60 | | 100 | |
| [wt %] | C18:0-PHS20:0 | | 80 | 80 | 20 | | | 100 |

| | | | | | | | |
|---|---|---|---|---|---|---|---|
| Total [wt %] | 100 | 100 | 100 | 100 | 100 | 100 | 100 |
| Melting point [° C.] | 120.6 | 123.9 | 120.8 | 120.0 | 122.0 | 124.2 | 126.2 |

<4> Evaluation of Recovery of Skin Barrier Function by Composition Containing PHC Using Isolated Cadaver Skin Based on Transepidermal Water Loss (TEWL)

<4-1> Preparation of PHC Mixture

A PHC mixture having the composition shown in Table 3 was obtained. The PHC mixture or each PHC species contained therein can be reproducibly prepared by, for example, the method exemplified above.

TABLE 3
Composition of PHC mixture

| PHC species | Ratio [wt %] |
|---|---|
| C18:0-PHS16:0 | 70.0 |
| C18:0-PHS18:1 | 0.7 |
| C18:0-PHS18:0 | 21.7 |
| C18:0-PHS20:1 | 0.3 |
| C18:0-PHS20:0 | 7.3 |

<4-2> Preparation of Compositions

The PHC mixture obtained in <4-1> or Ceramide III (Evonik) was mixed with fatty acids and cholesterol in amounts shown in Table 4, to prepare lipid mixtures. Ceramide III (Evonik) is a single kind of PHC species, C18:0-PHS18:0. Each lipid mixture was dissolved in chloroform/methanol (chloroform:methanol=2:1) at a concentration of 1 wt %, to prepare each sample.

TABLE 4
Composition of lipid mixtures

| | | |
|---|---|---|
| Sample No. | 14 | 15 |

| | | | |
|---|---|---|---|
| Amount | Fatty acids (Total) | 4.74 | 4.74 |
| [mg] | Palmitic acid | 0.06 | 0.06 |
| | Stearic acid | 0.16 | 0.16 |
| | Arachidic acid | 0.33 | 0.33 |
| | Behenic acid | 2.23 | 2.23 |
| | Lignoceric acid | 1.96 | 1.96 |
| | Cholesterol | 5.30 | 5.30 |
| | PHC mixture | 7.88 | 0 |
| | Ceramide III | 0 | 7.88 |

<4-3> Measurement of Transepidermal Water Loss (TEWL)

<4-3-1> Pre-Treatment of Skin

Isolated cadaver skin (KAC, frozen, Full-thickness) was thawed at a room temperature, immersed in chloroform/methanol (chloroform:methanol=2:1, the same shall apply to the following procedure), and left to stand for 5 min. The skin was taken out from chloroform/methanol, and adipose tissues were removed from the skin with a scalpel or scissors. The skin was immersed in a 0.5% SLS (sodium lauryl sulfate) aqueous solution, and shaken for 30 min. The skin was taken out from the SLS aqueous solution, immersed in distilled water, and shaken for 30 min. The skin was removed from the water, immersed in new distilled water, and immediately removed therefrom. The moisture on the surface of the skin was removed with a paper, such as Kimtowel. The skin was immersed in chloroform/methanol, and left to stand for 60 min. The skin was removed from chloroform/methanol. The skin was put on a soft paper wet with distilled water with the dermis side downward (i.e. with the dermis side directed to the paper), and left to stand for 60 min, to be air-dried.

<4-3-2> Determination of Application Amounts

Experiments for determining application amounts of samples were carried out using the pre-treated skin obtained in <4-3-1>. Each sample in a volume of 50 μl, 40 μl, or 30 μl was dropped to the stratum corneum side of the skin, and air-dried. Aluminum foil was placed on a block heater of 80° C., and the skin was placed thereon with the stratum corneum side downward, heated for 2 min, so that the sample was melted and permeated the skin. During the heating, a container of ice water was placed on the upper side (i.e. the dermis side) of the skin. After the heating, the skin and the aluminum foil were visually observed, and the maximum application amount at which the sample hardly remains on both the skin and the aluminum foil was determined for each sample.

<4-3-3> Measurement of Transepidermal Water Loss (TEWL)

A 24-well plate (Corning, Product Number 353047) was placed on the stratum corneum side of the pre-treated skin obtained in <4-3-1>, and they were fastened with a rubber band. A weight was further placed thereon, and they were left to stand for 2 hours, so that circle patterns each having a diameter of approximately 1.6 cm were impressed on the stratum corneum. The 24-well plate was separated from the skin. Each sample in the maximum application amount determined in <4-3-2> was dropped to the impressed circle pattern on the stratum corneum side of the skin, and air-dried. Aluminum foil was placed on a block heater of 80° C., and the skin was placed thereon with the stratum corneum side downward, heated for 2 min, so that the sample was melted and permeated the skin. During the heating, a container containing ice water was placed on the upper side (i.e. the dermis side) of the skin. After the heating, the skin was put on a paper wet with distilled water with the dermis side downward (i.e. with the dermis side directed to the paper), and they were put into a sealed vessel and left to stand for 16 hours or more at 30° C. The skin was removed from the sealed vessel, put on a paper wet with distilled water on a block heater of 26° C. located in a room of 20° C., and left to stand for 90 min. The transepidermal water loss (TEWL) of the skin was measured with Tewameter TM300 (Courage+Khazaka) and MPA580 (Courage+Khazaka). TEWL of the skin was calculated as a relative value based on the value for an untreated portion of the skin, which was taken as 100%. The term “untreated portion” referred to herein refers to a portion to which no sample was applied.

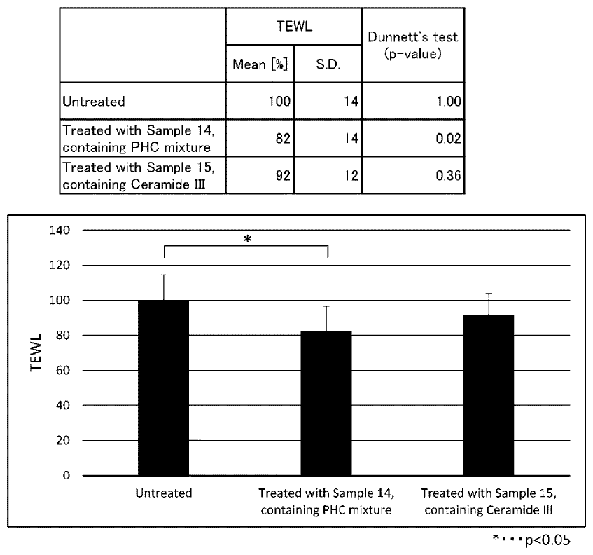

The results are shown in FIG. 1. TEWL of the portion treated with the sample 14, containing the PHC mixture, was significantly lower than that of the untreated portion, while TEWL of the portion treated with the sample 15, containing Ceramide III (i.e. C18:0-PHS18:0), was not significantly different from that of the untreated portion. In other words, the skin barrier function was significantly improved (recovered) by application of the sample 14. That is, again, it was indicated that a combined use of multiple PHC species having different lengths of alkyl chains improves a property of a composition containing the same as compared with when using a single kind of PHC species solely.

### INDUSTRIAL APPLICABILITY

According to the present invention, a composition, such as a cosmetic composition, can be provided.

We claim:

1. A composition, comprising two or more phytoceramides each having different lengths of alkyl chains.

2. The composition according to claim 1, wherein the two or more phytoceramides consist of:
(A) a phytoceramide (A) having a C16 phytosphingosine moiety in an amount of X % by weight to the total amount of the phytoceramides (A), (B), and (C);
(B) a phytoceramide (B) having a C18 phytosphingosine moiety in an amount of Y % by weight to the total amount of the phytoceramides (A), (B), and (C); and
(C) a phytoceramide (C) having a C20 phytosphingosine moiety in an amount of Z % by weight to the total amount of the phytoceramides (A), (B), and (C);
wherein at least two of X, Y, and Z are more than 0, and the sum of X, Y, and Z are less than 100.

3. The composition according to claim 1, wherein the composition is a cosmetic or pharmaceutical composition.

4. The composition according to claim 1, wherein the composition further comprises an ingredient typically used for formulating a cosmetic or pharmaceutical.

5. The composition according to claim 4, wherein the ingredient typically used for formulating a cosmetic or pharmaceutical is selected from the group consisting of an excipient, binder, disintegrant, lubricant, stabilizer, diluent, surfactant, pH adjuster, vitamin, mineral, perfume, pigment, preservative, terpenoid, lipid, fatty acid, alcohol, water, antioxidant, anti-inflammatory, moisturizing ingredient, anti-ageing ingredient, anti-cellulite ingredient, skin whitening ingredient, skin tanning ingredient, UV filter, and combinations thereof.

6. The composition according to claim 5, wherein the ingredient typically used for formulating a cosmetic or pharmaceutical is selected from the group consisting of a terpenoid, lipid, fatty acid, and combinations thereof.

7. The composition according to claim 5, wherein the terpenoid is selected from the group consisting of cholesterol, cholesterol sulfate, squalene, pristane, and combinations thereof.

8. The composition according to claim 5, wherein the lipid is selected from the group consisting of triolein, phosphatidylethanolamine, and combinations thereof.

9. The composition according to claim 5, wherein the fatty acid is selected from the group consisting of lauric acid, myristic acid, palmitic acid, stearic acid, arachidic acid, behenic acid, lignoceric acid, oleic acid, linolenic acid, and combinations thereof.

10. The composition according to claim 2, wherein X is 0-90, Y is 0-95, and Z is 0-50.

11. The composition according to claim 2, wherein X is 65-90, Y is 5-30, and Z is 1-20.

12. The composition according to claim 2,
wherein the phytoceramide (A) comprises a phytoceramide having a C16:0 phytosphingosine moiety,
wherein the phytoceramide (B) comprises a phytoceramide having a C18:0 phytosphingosine moiety, and
wherein the phytoceramide (C) comprises a phytoceramide having a C20:0 phytosphingosine moiety.

13. The composition according to claim 2,
wherein the phytoceramide (A) has an acyl chain having a length of C14 to C26,
wherein the phytoceramide (B) has an acyl chain having a length of C14 to C26, and
wherein the phytoceramide (C) has an acyl chain having a length of C14 to C26.
